(19) United States
(12) Patent Application Publication
(10) Pub. No.: US 2007/0162115 A1
(43) Pub. Date: Jul. 12, 2007

(54) Fixation ring for endocapsulary or extracapsulary prosthetic reconstruction in the anterior section region of the human eye

(76) Inventor: Heino Hermeking, Wuppertal (DE)

Correspondence Address: Thomas M Galgano;Galgano & Burke, 300 Rabro Drive, Suite 135, Hauppauge, NY, 11788, US

(21) Appl. No.: 10/557,927

(22) PCT Filed: May 20, 2003
(86) PCT No.: PCT/DE03/01649

§ 371 Date: Jan. 4, 2007

(51) Int. Cl. A61F 2/14

(52) U.S. Cl. 623/4.1

(57) ABSTRACT

The fixation ring has a flat annular body that has a central circular aperture (10). Two haptic stirrups (18) that lie opposite one another are attached to the annular body, which are angled upward from the ring plane, and outwards parallel to it. The annular body can be folded in half, and thereby reduced in its outside dimensions. It is suitable for unfolding into its flat original position again by means of its inherent elasticity.

The invention relates to a fixation ring for endocapsulary or extracapsulary prosthetic reconstruction in the anterior section region of the human eye.

Intraocular prostheses primarily fulfill optical tasks. They generally serve to replace the human lens after surgery to remove cataracts, as a diopter link in the optical chain of the eye.

The following possibilities exist for fixation of an intraocular prosthesis in the eye (from anterior to posterior):
1. chamber-angle supported or chamber-angle fixed; anterior chamber lens;
2. iris-enclaved, i.e. iris-fixed; artisan lens;
3. iris-supported, i.e. fixed by means of the pupil; iris clip lens;
4. sulcus-positioned, i.e. in the case of the presence of sufficient remaining capsule material and appropriate suspension (zonulae), embedding of the corresponding lens haptics in the sulcus ciliaris;
5. endocapsulary positioning, i.e. introduction of the intraocular lens in the lens capsule bag;
6. sclera fixation of the intraocular prosthesis, i.e. trans-scleral suture and anchoring of the lens haptic.

In general, endocapsulary positioning is preferred, but this does presuppose an essentially intact lens capsule bag with corresponding suspension. In the case of a lack of presence of the capsule bag, other fixation techniques for alternative positioning of the prosthesis are possible, whereby the individual positioning types are encumbered with different risk factors. For example, in the case of chamber-angle-supported prostheses, decompensation of the cornea can develop.

A fixation ring for one or more diaphragm(s), with which an artificial pupil opening is created, is known from WO 98/56 314 A1, as part of an endocapsulary prosthesis system. The fixation ring has a flat annular body that has a central circular aperture, on which two haptic stirrups that lie opposite one another are attached, which are angled upward from the ring plane, and outwards parallel to it. The haptic stirrups serve to anchor the diaphragm(s). The central aperture of the fixation ring can be used for attaching an optical prosthesis.

The individual properties of the capsule bag, such as equator dimensions, for example, have the result that at given dimensions of the diaphragm(s), for example a specific outside diameter, an aperture to the center of the prosthetic structure is implemented, which is characterized by the individual outside diameters determined by the anatomical conditions. If, for example, the equator of the lens capsule is smaller than the outside diameter of the prosthesis, the aperture is made smaller and no longer corresponds to the circular default. For this reason, it is practical to introduce the fixation ring, which creates a standardized pupil size, as the central prosthetic element. Fundamentally, the outside diameter of the entire prosthetic structure should not be larger than the capsule bag equator, in this connection.

In practice, it has proven to be difficult to insert the fixation ring into the prosthetic aperture of the diaphragm(s). It is the task of the invention to create a fixation ring that can be inserted more easily into other prosthetic parts.

In the case of the fixation ring that accomplishes this task, the annular body can be folded in half, and is suitable for unfolding into its flat original position again by means of its inherent elasticity.

The annular body of the fixation ring has shape memory and an elastic shape stability. Because it can be folded in half, controlled insertion into an inner, i.e. centrally situated aperture, which differs in its dimensions depending on the outside diameter, of a prosthetic outer structure is possible. The fixation ring that can be folded in half is an important element for standardizing and stabilizing the prosthetic configuration.

An optic can be introduced, particularly inserted or clipped into the aperture of the fixation ring. The fixation ring can also serve as a holder for a diopter element in another manner, which element is well positioned by means of the fixation ring.

The fixation ring according to the invention is used for endocapsulary prosthesis systems that serve to construct a diaphragm that is used to replace iris absence or iris defects, i.e. iris deficiencies. The fixation ring is suitable for replacing the fixation ring according to WO 98/56 314 A1. In this connection, because of its ability to be folded in half, it offers significant advantages in terms of handling.

In the case of one preferred embodiment, the annular body of the fixation ring is divided in the center and provided with lateral connecting stirrups that lie opposite one another, which are elastic and bridge the division. The annular body can be folded in half at the division. Thanks to the elastic connecting stirrups, it springs back into its flat original position.

In the case of one preferred embodiment, the annular body of the fixation ring is essentially circular, and widens around the connecting stirrups that project away laterally.

In the case of one preferred embodiment, the connecting stirrups take up approximately one eighth of the circumference of the annular body. The connecting stirrups have a narrow, rounded rectangular U shape.

In the case of one preferred embodiment variant, the haptic stirrups are situated between the connecting stirrups.

In the case of one preferred embodiment, the haptic stirrups start approximately in the radial center of the annular body, on both ends. Their part that extends parallel to the ring plane is curved. In the center, it reaches to the outer edge of the annular body, or it projects slightly outwards beyond it.

In the case of one preferred embodiment, the fixation ring has dual symmetry of rotation.

In the case of one preferred embodiment, the fixation ring consists of dyed polymethyl methacrylate (PMMA) or polycarbonate. Despite the rigidity of these materials, the fixation ring can be folded in half because of its geometric configuration.

The invention will be explained in greater detail in the following, using exemplary embodiments shown in the drawing. This shows:

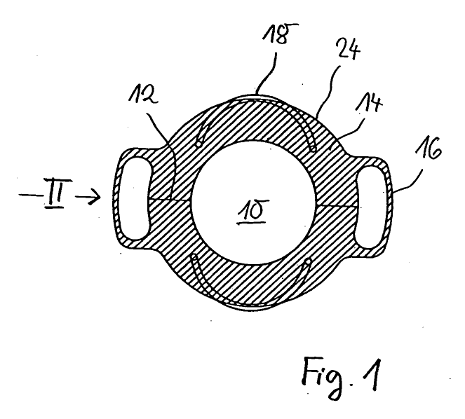

FIG. 1 a top view of a fixation ring;

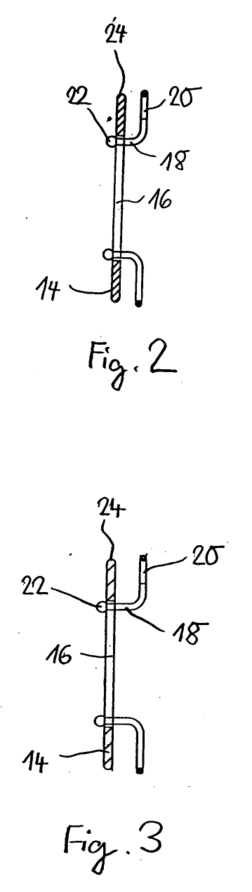

FIG. 2 a side view of the fixation ring with a view in the direction II of FIG. 1; and

FIG. 3 a side view of the fixation ring in an alternative embodiment.

The fixation ring consists of dyed polymethyl methacrylate (PMMA) or polycarbonate. It has a flat annular body essentially in the shape of a circular ring having an outside diameter of 5 mm and a central circular aperture 10 that has a diameter of 3 mm.

The annular body is divided in the middle, along a diagonal, straight dividing line 12. Its halves 14 are connected with connecting stirrups 16 that are molded onto the annular body and project away from it, which bridge the dividing line 12. The connecting stirrups 16 lie in the plane of the annular body. They attach to the halves 14 of the annular body with mirror symmetry to the dividing line 12. The fixation ring can be folded along the dividing line 12 and thereby can be reduced in its outside dimensions. The connecting stirrups 16 are so elastic that the fixation ring tends to spring back into its flat original position, which is shown in FIG. 1. In this position, the half rings 14 abut flush with one another, so that they complement one another to form a planar full ring.

Haptic stirrups 18 attach to the half rings 14 between the connecting stirrups 16. The haptic stirrups 18 are angled away out of the ring plane, vertically upward, at both ends. Their stirrup backs 20 run parallel to the ring plane, at the same height. The attachment points 22 of the haptic stirrups 18 are situated approximately in the radial center of the half ring 14, in each instance. The stirrup back 20 is curved outwards. It projects slightly beyond the outer edge 24 of the annular body.

The fixation ring has dual symmetry of rotation. It makes a transition into itself when turned by 180° about the center of the aperture 10.

Compared with the fixation ring according to FIG. 1 and FIG. 2, the haptic stirrups 18 are set out further from the ring plane in the case of the fixation ring according to FIG. 3. Therefore more of a prosthesis fits between the annular body and the haptic stirrups of the fixation ring.

### LIST OF REFERENCE SYMBOLS

10 aperture
12 dividing line
14 half ring
16 connecting stirrup
18 haptic stirrup
20 stirrup back
22 attachment point
24 edge

1. Fixation ring for endocapsulary or extracapsulary prosthetic reconstruction in the anterior section region of the human eye, using a flat annular body that has a central circular aperture, on which two haptic stirrups that lie opposite one another are attached, which are angled upward from the ring plane, and outwards parallel to it, wherein said annular body can be folded in half, and is suitable for unfolding into its flat original position again by means of its inherent elasticity.

2. Fixation ring according to claim 1, wherein said annular body is divided in the center and provided with lateral connecting stirrups that lie opposite one another, which are elastic and bridge the division.

3. Fixation ring according to claim 2, wherein said annular body is essentially circular, and widens around the connecting stirrups that project away laterally.

4. Fixation ring according to claim 2, wherein said connecting stirrups take up approximately one eighth of the circumference of the annular body, and have a narrow, rounded rectangular U shape.

5. Fixation ring according to claim 2, wherein said haptic stirrups are situated between the connecting stirrups.

6. Fixation ring according to claim 2, wherein said haptic stirrups start approximately in the radial center of the annular body, on both ends, and that their part that extends parallel to the ring plane is curved and in the center, it reaches to the outer edge of the annular body, or it projects slightly outwards beyond it.

7. Fixation ring according to claim 1, wherein it has dual symmetry of rotation.

8. Fixation ring according to claim 1, wherein its aperture has a diameter of approximately 3 mm.

9. Fixation ring according to claim 1, wherein it consists of dyed polymethyl methacrylate (PMMA) or polycarbonate.
